(19) United States
(12) Patent Application Publication
(10) Pub. No.: US 2001/0000228 A1
(43) Pub. Date: Apr. 12, 2001

(54) Use of a modified baculovirus containing exogenous nucleic acid for the manufacture of a medicament for delivering said nucleic acid to the hepatocytes

(75) Inventors: Michael J. McGarvey, London (GB); Howard C. Thomas, London (GB)

Correspondence Address: Nixon & Vanderhye P.C., 1100 N. Glebe Rd., 8th Floor, Arlington, VA, 22201, US

(73) Assignee: IMPERIAL COLLEGE INNOVATIONS LIMITED

(21) Appl. No.: 09/729,856

(22) Filed: Dec. 6, 2000

Related U.S. Application Data
(63) Continuation of application No. 09/428,532, filed on Oct. 28, 1999, now abandoned.

(30) Foreign Application Priority Data
Apr. 29, 1997 (GB) 9708698.7
Apr. 29, 1998 (GB) PCT/GB98/01249

(51) Int. Cl. A61K 31/70; A01N 43/04; A61K 48/00; A01N 63/00

(52) U.S. Cl. 424/93.21; 514/44

(57) ABSTRACT

Modified baculovirus particles containing prophylactic and/or therapeutic nucleic acid are provided, together with their use in treating liver conditions such as viral infections.

1. The present invention relates to materials for use in gene therapy and novel methods of gene therapy. In particular, it relates to novel methods of delivering selected nucleic acid to hepatocytes in order to treat viral infections of such cells, for instance DNA encoding an effector enzyme capable of converting a prodrug to its active form.

2. Hepatitis viruses, e.g. hepatitis B (HBV) and hepatitis C (HCV), represent a major health problem on a worldwide scale. In the case of HBV, there are 350 million cases of chronic infections. Of these, 10-20% will develop into cases of cirrhosis. HBV is also a major cause of primary liver cancer. For HCV, there are around 200 million chronic infections worldwide. Once again, 10-20% will result in cirrhosis, and once again the virus is a cause of primary liver cancer.

3. Although conventional drug therapies have been, and are being, developed to combat HBV and HCV, such therapies are by no means successful in all cases. For example, Interferon α has been developed as a therapy against HBV but is generally only effective in around 30% of cases. Other drug-based therapies include the use of compounds, which can interfere with the viral replication cycle, i.e. by interfering with synthesis of viral nucleic acid or the maturation of viral proteins by viral proteases. However, such compounds are themselves quite toxic towards uninfected cells and therefore represent a non-specific treatment. There is thus a continuing need to develop novel treatments for hepatitis viral infections which in particular can be targeted to infected cells, i.e. liver cells.

4. In recent years, with the continuing identification of specific genes responsible for certain disease conditions, the concept of “gene therapy” has attracted a great deal of attention. The potential to deliver a new gene, or even part of a gene sequence, to a defective cell in order to correct such an inherent deficiency is an attractive one. The approach is also potentially attractive as a means of targeting particular cells in order to obtain expression of, for example, a therapeutic protein in those cells.

5. Thus, gene therapy represents a potential method of targeting specific cells, e.g. liver cells. A working system could provide various options for interfering with the replication cycle of viruses, particularly those which can cause chronic infections such as HBV and HCV. Alternatively, the system could be used to deliver nucleic acid encoding therapeutic agents which when expressed within liver cells will protect them against the viruses.

6. Various gene delivery systems have been proposed for targeting of liver cells. They include the use of Adenoviruses, Retroviruses, liposomes and receptor targeting, e.g. via the asialoglycoprotein receptor. All of these systems have significant disadvantages when used as gene targeting and delivery systems. Although adenoviruses can deliver nucleic acid encoding the therapeutic agent to liver cells, they can also infect other cells and deliver the nucleic acid to them. In addition, they can elicit a strong cell mediated immune response and the virus itself can have cytopathic effects. Retroviruses usually have low virus titres making it difficult to deliver a large enough dose of the effector gene(s). In addition, the integration of the retrovirus proviral DNA into the genome of the target cell could alter the pattern of gene expression within the cell and this could have deleterious effects on the cell. Currently liposomes are relatively non-specific in their uptake by liver cells and this could lead to the delivery of the nucleic acid encoding the therapeutic agent to non-target cells. Although the asialoglycoprotein receptor is liver specific, and can consequently direct the uptake of effector genes coupled to the asialoglycoprotein ligand (ASGPL), this system for gene delivery involves relatively complex chemical processes to couple the effector genes to the ASGPL. This involves the use of spacer molecules e.g. peptides.

7. We have now developed an alternative system based on Baculoviruses. These are insect viruses which have shown hepatocyte specificity (Hofman et al, PNAS 92:10099-10103 (1995); Boyce and Bucher, PNAS, 93:2348-2352 (1996)). Thus, although these viruses can “infect” liver cells, they do not replicate and so should represent less risk as compared to retroviruses for instance. Another key feature of Baculoviruses is that they have a large potential genetic “payload”, around 15 kB; i.e. up to 15 kB of the baculovirus genome can be replaced by nucleic acid which encodes therapeutic agents.

8. Thus, in a first aspect, the present invention provides the use of a Baculovirus containing therapeutic and/or prophylactic nucleic acid to deliver said nucleic acid to one or more hepatocytes. In the context of the present invention, “therapeutic nucleic acid” includes DNA or RNA whose expression is capable of directly or indirectly treating an infection in the hepatocytes or providing a prophylactic effect. Suitably, the therapeutic nucleic acid is directed against viral infections, particularly HBV and HCV infections.

9. Generally, the nucleic acid will be provided in the form of a nucleic acid construct which will also comprise regulatory nucleic acid, e.g. a suitable promoter sequence, to provide for expression of the therapeutic/prophylactic nucleic acid in the target cell(s). For instance a mammalian promoter can be used, examples of which include the CMV and SV40 promoters. Suitably, the construct will be provided in the form of a vector, e.g. a plasmid construct, which in turn will be “packaged” by the baculovirus. The packaging of the nucleic acid which encodes a therapeutic/prophylactic agent into baculoviruses is carried out by recombination between the polyhedrin gene in baculovirus DNA and baculovirus polyhedrin sequences that form part of the recombinant plasmid or “shuttle” vector in which the nucleic acid which encodes a therapeutic/prophylactic agent has been cloned. The result of this recombination event is the production of a recombinant baculovirus which contains the nucleic acid which encodes a therapeutic/prophylactic agent as an integral part of the baculovirus genomic DNA.

10. The present invention provides for several broad approaches. Firstly, the baculovirus system can be used to deliver a gene encoding an enzyme for instance which can convert a prodrug compound to its active form. An example of such an approach would be the delivery of a gene encoding a (TK) enzyme, eg. the Herpes Simplex Virus (HSV) thymidine kinase, which is capable of converting ganciclovir or araM to their active forms. In this way, liver cells of a subject can be “enabled” with respect to this conversion which should result in therapeutically effective levels of active drug being formed in the liver upon administration of ganciclovir or araM.

11. This embodiment of the invention can be further refined by the inclusion in the construct of one or more regulatory sequences which is/are only activated in the presence of the infective agent against which the therapy/prophylaxis is directed. Examples of such an approach would include the use of viral promoters in the construct which would lead to expression only in the presence of the target virus (where such a promoter is only recognised by a viral nucleic acid polymerase encoded by the virus itself). Another example would be to utilise a specific activator, such as the HBV X protein. This is a transactivator protein capable of “switching on” certain control sequences, e.g. the NFκB sequence (Lucito and Schnieder, J Virol. 66 983-991 (1992); Chirillo et al., J Virol. 70 641-646 (1996)). Thus, if this control sequence is coupled with the coding sequence for TK, expression of the TK gene will be switched on in the presence of HBV, allowing conversion of ganciclovir or araM to active drug in those cells infected with HBV.

12. To improve the spread of baculovirus encoded recombinant proteins to liver cells adjacent to cells that have been infected with a recombinant baculovirus, fusion proteins of Herpes simplex virus (HSV) protein VP22 with CAT and HSV-TK, encoded by such a baculovirus should allow these proteins to spread from baculovirus infected to uninfected cells since it has been shown that the Herpes simplex virus (HSV) protein, VP22, can spread from cell to cell very efficiently when the gene for this protein is transfected into cells (Elliot G and O'Hare P (1997) Cell 88: 223-233).

13. An alternative approach is based on the use of “antisense” nucleic acid. This is nucleic acid which when transcribed results in a nucleic acid molecule which is complementary to at least part of the genome of an infective agent, such as a virus. This complementary nucleic acid will thus bind to the nucleic acid of the infective agent, e.g. a hepatitis virus, and will prevent its transcription and ultimate translation. Thus, for instance, the construct of the invention could encode antisense nucleic acid directed against the HBV replicase gene. Prevention of transcription of this gene will therefore prevent the formation of new viral particles. Similarly, the antisense nucleic acid could be directed against the HBV capsid protein or envelope protein genes. Ribozymes, which contain antisense RNA coupled to RNA domains capable of catalytically cleaving target RNAs and which are specifically designed to degrade HBV and HCV viral genomic and/or messenger RNAs (Lieber et al., J Virol. 70 8782-8791 (1996)) can also be encoded in these recombinant baculoviruses.

14. As mentioned above, the present invention can also be used to deliver therapeutic agents directly. One example of such an approach would be a construct encoding a particular interferon-α subtype for use in the treatment or prophylaxis of a hepatitis virus infection. It is known for instance that certain interferon-α subtypes are particularly effective against viral infections in certain cell types. Thus, in WO-A-9524212 it is disclosed that IFNα₈ is particularly effective against viral infection of liver cells. Thus, a construct of the invention encoding IFNα₈, and under the control of a selective expression system as described above, would provide a defence against HBV for instance which would be activated by the presence of the virus.

15. Another example of the direct delivery of therapeutic agents would be a construct encoding interleukin-12 (IL-12) in the treatment or prophylaxis of a hepatitis virus infection. It is known that IL-12 is particularly effective against viral infections in certain cell types IL-12 has been shown to shift the Th2 mediated immune response to HBV e-antigen to a Th1 response. There is evidence that this Th2 to Th1 shift may favour viral clearance of in chronic HBV infection (Milich et al., Proc. Nat'l. Acad. Sci. USA 92 6847-6851 (1995)).

16. The delivery of genes which can convert a prodrug to its active form capable of killing liver tumour cells can be achieved with the baculovirus gene delivery system described. Baculoviruses have been shown to be able to deliver genes to the liver tumour cell lines HepG2 and Huh7 and that when regulated by mammalian promoters these genes can be expressed in these tumour cell lines (Hofmann C, et al. Proc Natl Acad Sci USA 92: 10099-10103. (1995); Boyce F M, Bucher NLR Proc Natl Acad Sci USA 93: 2348-2352 (1995)). The expression of the HSV TK gene can convert ganciclovir to its active form which is capable of killing tumour cells. By regulating the expression of the HSV TK gene in a baculovirus vector sequencing such as promoter and enhancer elements that can be activated in liver tumour cells but not in adult liver cells (a good example are the promoter and enhancer elements from the alpha-foetoprotein gene) it should be possible to restrict the expression of the HSV TK gene and the activation of ganciclovir to liver tumour cells and not normal hepatocytes, thus increasing the specificity of the anti-tumour therapy.

17. To monitor the expression of therapeutic genes in liver cells, in vivo, it would be very helpful to have a non-invasive means of monitoring the expression of such coding sequences which were introduced into liver by the use of recombinant baculoviruses. Human creatine kinase can increase the phosphorylation levels of its substrate, creatine, and this alteration in phosphorylation can be detected by magnetic resonance spectroscopy (Menon et al, Hepatology 21 417-427 (1995)). Liver cells do not express creatine kinase. Therefore the introduction of creatine kinase into liver by the use of baculoviruses that express the creatine kinase coding sequence (Perryman et al, BBRC 140 981-989; Kaye et al, J Clin. Invest. 79 1412-1420 (1987)) should allow levels of expression of therapeutic proteins which are co-expressed in the same baculoviruses to be monitored.

18. In a second aspect, the present invention provides a baculovirus containing therapeutic and/or prophylactic nucleic acid. Such a baculovirus can have any one or more of the features as discussed herein.

19. Thus, the baculovirus can be used in the manufacture of a medicament for use in the treatment or prophylaxis of an infection, e.g. a viral infection such as HBV or HCV, or for use in the prophylaxis or treatment of liver cancer and form further aspects of the invention.

20. In a further aspect the present invention provides a pharmaceutical formulation comprising a baculovirus as defined in claim 21 or claim 22 optionally together with one or more pharmaceutically acceptable carriers, excipients and/or diluents. This pharmaceutical composition will generally be provided in a sterile unit dosage form in a sealed container.

21. Pharmaceutical compositions within the scope of the present invention may include one or more of the following: preserving agents, solubilising agents, stabilising agents, wetting agents, emulsifiers, sweeteners, colorants, odourants, salts, buffers, coating agents or antioxidants. They may also contain therapeutically active agents in addition to the modified baculovirus of the present invention.

### Routes of Administration

22. The pharmaceutical compositions within the scope of the present invention may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such a composition may be prepared by any method known in the art of pharmacy, for example by admixing the active ingredient with a carrier under sterile conditions.

23. Various routes of administration will now be considered in greater detail:

24. (i) Oral Administration

25. Pharmaceutical compositions adapted for oral administration may be provided as capsules or tablets; as powders or granules; as solutions, syrups or suspensions (in aqueous or non-aqueous liquids); as edible foams or whips; or as emulsions.

26. Tablets or hard gelatine capsules may comprise lactose, maize starch or derivatives thereof, stearic acid or salts thereof.

27. Soft gelatine capsules may comprise vegetable oils, waxes, fats, semi-solid, or liquid polyols etc.

28. Solutions and syrups may comprise water, polyols and sugars. For the preparation of suspensions oils (e.g. vegetable oils) may be used to provide oil-in-water or water-in-oil suspensions.

29. (ii) Transdermal Administration

30. Pharmaceutical compositions adapted for transdermal administration may be provided as discrete patches intended to remain in intimate contact with the epidermis of the recipient for a prolonged period of time. For example, the active ingredient may be delivered from the patch by iontophoresis (Iontophoresis is described in Pharmaceutical Research, 3(6):318 (1986)).

31. (iii) Topical Administration

32. Pharmaceutical compositions adapted for topical administration may be provided as ointments, creams, suspensions, lotions, powders, solutions, pastes, gels, sprays, aerosols or oils.

33. Pharmaceutical compositions adapted for topical administration in the mouth include lozenges, pastilles and mouthwashes.

34. (iv) Rectal Administration

35. Pharmaceutical compositions adapted for rectal administration may be provided as suppositories or enemas.

36. (v) Nasal Administration

37. Pharmaceutical compositions adapted for nasal administration which use solid carriers include a coarse powder (e.g. having a particle size in the range of 20 to 500 microns). This can be administered in the manner in which snuff is taken, i.e. by rapid inhalation through the nose from a container of powder held close to the nose.

38. Compositions adopted for nasal administration which use liquid carriers include nasal sprays or nasal drops. These may comprise aqueous or oil solutions of the active ingredient.

39. Pharmaceutical compositions adapted for administration by inhalation include fine particle dusts or mists, which may be generated by means of various types of apparatus, e.g. pressurised aerosols, nebulisers or insufflators. Such apparatus can be constructed so as to provide predetermined dosages of the active ingredient.

40. (vi) Vaginal Administration

41. Pharmaceutical compositions adapted for vaginal administration may be provided as pessaries, tampons, creams, gels, pastes, foams or spray formulations.

42. (vii) Parenteral Administration

43. Pharmaceutical compositions adapted for parenteral administration include aqueous and non-aqueous sterile injectable solutions or suspensions. These may contain antioxidants, buffers, bacteriostats and solutes which render the compositions substantially isotonic with the blood of an intended recipient. Other components which may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilised) condition requiring only the addition of a sterile liquid carrier, e.g. sterile water for injections, immediately prior to use. Extemporaneous injection solutions and suspensions may be prepared from sterile powders, granules and tablets.

### Dosages

44. Dosages will be readily determinable by routine trials, and will be under the control of the physician or clinician. The guiding principle for determining a suitable dose will be delivery of a suitably efficacious but non-toxic, or acceptably toxic, amount of material.

45. The pharmaceutical formulations of the invention can be used in the treatment or prophylaxis of infections, such as viral infections as well as in methods for the prophylaxis treatment of liver cancer. Such methods form yet further aspects of the invention. In preferred embodiment the methods are for the treatment or prophylaxis of HBV or HCV.

46. The invention will now be described with reference to the following examples, which should not be construed as in any way limiting the invention.

### EXAMPLE

### Recombinant Baculoviruses containing the CAT Reporter Gene or the Herpes Simplex Virus Thymidine Kinase Gene

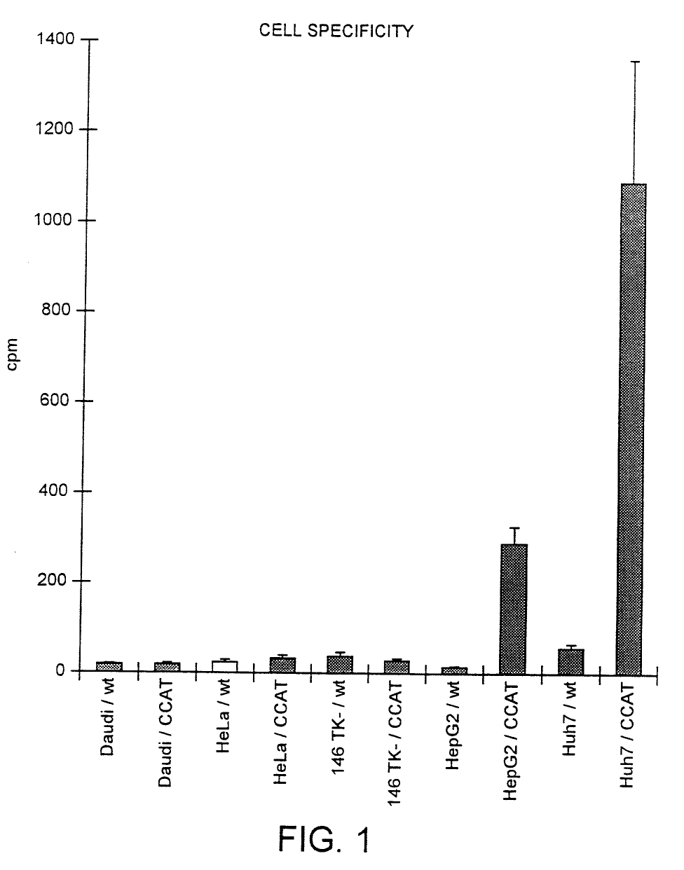

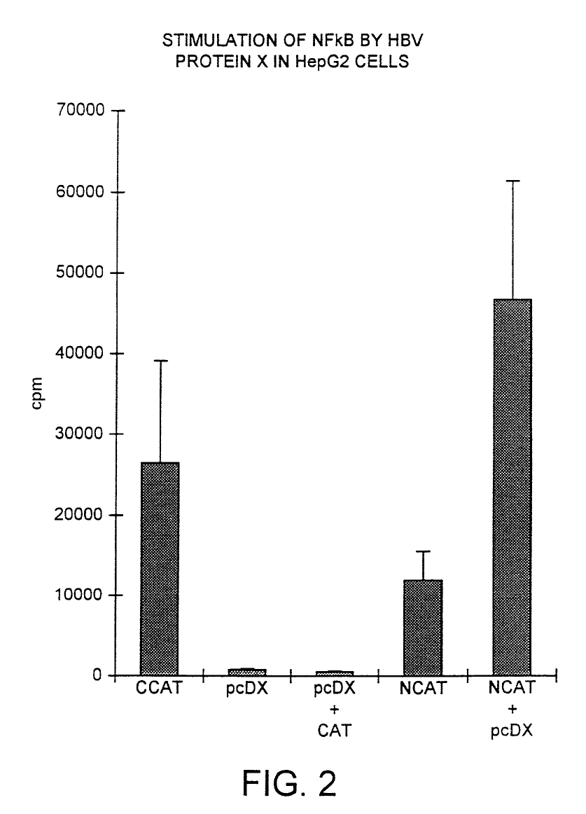

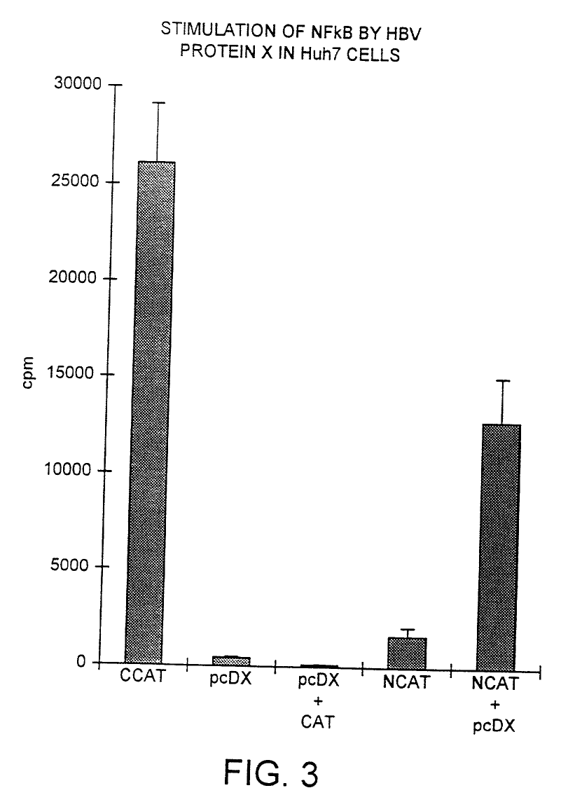

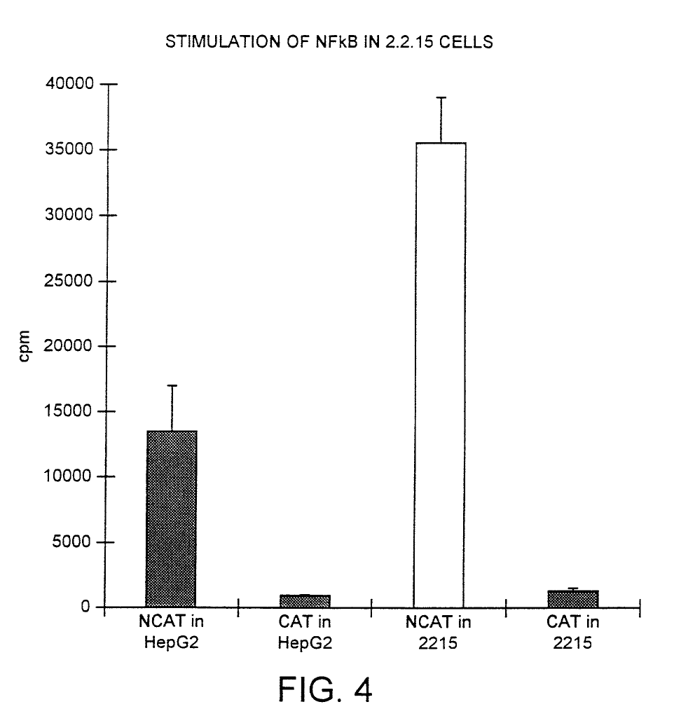

47. To develop the gene delivery system we have made recombinant baculoviruses which contain the chloramphenicol acetyl transferase (CAT) reporter gene, under the control of a baculovirus polyhedrin promoter (CAT), the CAT gene under the control of a CMV promoter, CCAT the CAT gene under the control of a NFκB promoter, NCAT and the thymidine kinase (TK) gene under the control of a NFκB promoter, NTK, using the shuttle vector pCRBac (Invitrogen Inc). The CAT coding sequence (CAT) was obtained from the plasmid pCDNA3.1 (InVitrogen Inc) by the polymerase chain reaction (PCR). To amplify the CAT coding sequence, forward and reverse primers of 20 nucleotides that corresponded to the 5′ end and 3′ end of the CAT coding sequence were used. The CAT DNA sequence was then ligated into the baculoviral shuttle vector pCRBac according to the procedure described in the Baculovirus TA Cloning Kit, to produce the plasmid pCAT. The DNA sequence that contained a CMV promoter at the 5′ end of the molecule and the CAT coding sequence at the 3′ end (CMV-CAT), was obtained from the plasmid pCDNA3.1 by using the polymerase chain reaction (PCR). To amplify the CMV-CAT DNA sequence, forward and reverse primers of 20 nucleotides that corresponded to the 5′ end of the CMV promoter and 3′ end of the CAT coding sequence were used. The CMV-CAT DNA was then ligated into pCRBac (as described above) to produce pCCAT. To produce a shuttle vector that contained the CAT coding sequence under the control of the NFκB promoter, the same PCR procedure (as described above) was used but with a forward primer which was 50 nucleotides in length that contained both a 30 nucleotide sequence that corresponded to the NFκB promoter and a 20 nucleotide sequence that corresponded to the sequence 50-70 nucleotides upstream of the CAT coding sequence in pCAT and a reverse primer that corresponds to the 3′ end of the CAT coding sequence. The NFκB-CAT DNA was then ligated into pCRBac (as described above) to produce pNCAT. To produce a shuttle vector that contained the TK coding sequence under the control of the NFκB promoter, the TK coding sequence was amplified from herpes simplex 1 (HSV1) DNA by the same PCR procedure (as described above) using a forward primer, which was 50 nucleotides in length and which was comprised of a 30 nucleotide sequence that corresponded to the NFκB promoter and a 20 nucleotide sequence that corresponded to the 5′ end of the TK coding sequence and a reverse 20 nucleotide primer that corresponded to the 3′ end of the TK coding sequence. HSV1 DNA was extracted from 10⁸ HSV1 particles according to a procedure previously described (Garson et al. Lancet 1 1419-1422 (1990)). The NFκB-TK DNA was then ligated into pCRBac (as described above) to produce pNTK. A plasmid was also made that expresses the HBV X protein coding sequence under the control of a CMV promoter by ligating the HBV X coding sequence into pcDNA3.1 to produce the plasmid pcDX. The HBV X coding sequence was obtained by PCR amplification using forward and reverse primers that corresponded to the 20 nucleotides at the 5′ and 3′ ends of the HBV X coding sequence from plasmid pHBV130.4 (Gough and Murray J. Mol. Biol. 162 43-67 (1982)). Recombinant baculoviruses were made with the shuttle plasmids described above with the Bac-N-Blue transfection kit (InVitrogen). All the procedures use to make these recombinant baculoviruses were as described in the Bac-N-Blue transfection kit. This resulted in baculoviruses that expressed, CAT under the control of the CMV promoter (CCAT), CAT under the control of the NFκB promoter (NCAT), and TK under the control of the NFκB promoter (NTK). These baculoviruses were used to infect tissue culture, hepatocyte cell lines Huh-7 and HepG2, HepG2 (2.2.15) cells (Acs G et al. PNAS 84: 4641-4644 (1987)), and non-hepatocyte cell lines HeLa, Daudi and 146 TK⁻. CAT activity was assayed for by using a CAT enzyme assay system (E100—Promega) and the procedure described in this assay system was followed. As shown in FIG. 1 the CAT activity of CCAT was only detectable in the hepatocyte cell lines but not in the non-hepatocyte cell lines, showing that baculoviruses are specific for liver cells. In hepatocyte cell lines infected by NCAT, the CAT activity levels increased more than five fold when the HBV X protein was co-expressed in these cells, showing that HBV can up regulate the expression levels of potential therapeutic agents by the procedures described. FIGS. 2 and 3 show the infection of HepG2 cells and Huh7 cells with either CCAT, NCAT, NCAT in cells which express the HBV-X protein (NCAT+pcDX) or CAT (no mammalian promoter) in cells which express the HBV-X protein (CAT+pcDX) and uninfected cells which express the HBV-X protein (pcDX). FIG. 4 shows a comparison of the stimulation of NCAT in HepG2.2.15 cells (which express the HBV-X protein constitutively) and HepG2 cells. In all three experiments the expression of the HBV-X protein shows a stimulatory effect on NCAT expression. Expression of the HBV X protein in these cells was achieved by transfection of pcDX into the cells by using Transfectin (Life Technologies) and the transfection procedure used was the one supplied with Transfectin.

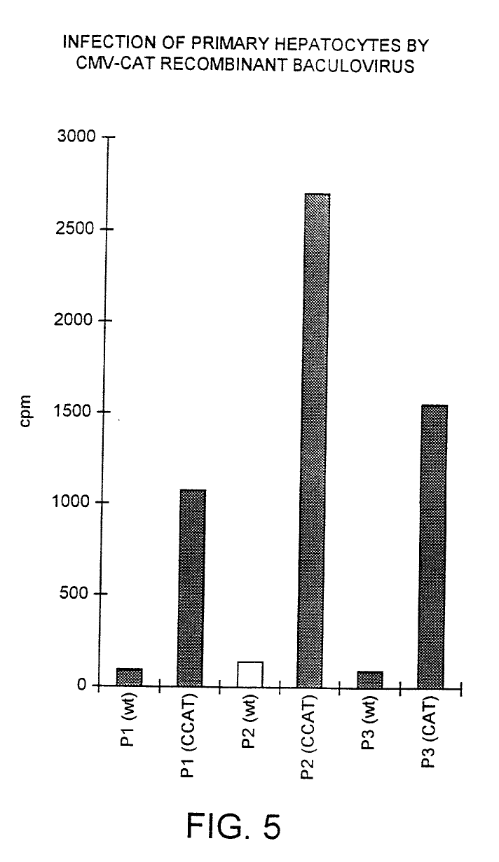

48. Baculovirus CCAT was used to infect human primary hepatocytes obtained from liver biopsies. These human primary hepatocytes were prepared by mincing human liver samples followed by digestion with collagenase. Briefly, the minced liver was washed five times with Phosphate buffered saline (PBS) and then a pre-warmed (to 37° C.) solution of 0.05% collagenase (Life Technologies) in PBS was added. The collagenase solution/liver mixture was stirred at 37° C. for five minutes. The solution which now contained single hepatocytes in suspension was then removed and placed in a container that contained complete cell culture medium RMPI with 10% bovine foetal serum to neutralise the collagenase and was then centrifuged (at 100×g) for 5 minutes to pellet the cells. The cells were then plated out and infected with CCAT. FIG. 5 shows the results with three biopsies from different livers (P1-P3). The results show that CCAT infection resulted in the expression of CAT whereas, the infection of the same numbers of cells from each biopsy with wild type baculovirus shows only background levels of CAT activity.

49. The TK activity of baculovirus NTK was measured by the use of a genetic complementation assay (Metzger C, et al (1994). J. Virol. 69: 8423-8427). Briefly, monolayers of HepG2 cells, which were also transfected with pcDX to express the HBV-X protein, were infected with baculovirus NTK. Cells were either grown in cell culture medium (RPMI plus 10% bovine foetal serum) or the same cell culture medium supplemented with ganciclovir to a final concentration of 1 mM. The cells were then infected with a recombinant thymidine kinase deficient (TK⁻) vaccinia virus (vvT7-3) which was allowed to grow in these cells for two days. The number of vvT7-3 vaccinia virus plaques obtained in the absence or presence of ganciclovir were quantified. There was a reduction in the number of vaccinia virus plaques by over 90% when the TK protein encoded by baculovirus NTK was expressed in these liver cells lines in the presence of ganciclovir.

50. NTK was shown to be active in vivo in woodchucks chronically infected with woodchuck hepatitis virus (WHV). Approximately 0.4 ml aliquots of NTK, containing about 10⁹ plaque forming units per ml were injected into the livers of WHV chronically infected woodchucks. Needle biopsies of the same regions of the woodchuck livers were stained with mouse monoclonal antibody RFHBe2 (to hepatitis B virus e antigen and has previously been shown to react with WHV infected liver cells). In control liver approximately 10% of the cell stained for WHV e-antigen whereas in the liver infected with NTK the number of cells staining for WHV e-antigen was reduced to 4%.

Claims:

1. The use of a Baculovirus containing therapeutic and/or prophylactic nucleic acid to deliver said nucleic acid to one or more hepatocytes.

2. The use as claimed in claim 1 wherein the nucleic acid is directed against a viral infection.

3. The use as claimed in claim 2 wherein the viral infection is HBV and/or HCV.

4. The use as claimed in any one of claims 1 to 3 wherein the nucleic acid encodes an enzyme capable of converting a prodrug to its active form.

5. The use as claimed in claim 4 wherein the gene encodes a thymidine kinase (TK) enzyme, eg. Herpes Simplex Virus (HSV) thymidine kinase.

6. The use as claimed in claim 4 or claim 5 wherein the nucleic acid comprises one or more regulatory sequences which are active in the presence of an infective agent.

7. The use as claimed in claim 6 wherein the one or more regulatory sequences include a viral promoter.

8. The use as claimed in claim 6 wherein the one or more regulatory sequences include nucleic acid encoding a specific activator, eg. the HBV X protein.

9. The use as claimed in any one of claims 1 to 3 wherein the nucleic acid includes antisense nucleic acid.

10. The use as claimed in claim 9 wherein the transcription product of the antisense nucleic acid is complementary to at least a part of the genome of an infection agent, eg. a virus.

11. The use as claimed in claim 10 wherein the transcription product of the antisense nucleic acid is complementary to at least a part of the HBV or HCV genome.

12. The use as claimed in claim 11 wherein the transcription product of the antisense nucleic acid is complementary to at least a part of the HBV replicase gene, the HBV capsid protein gene or one or more of the HBV envelope protein genes.

13. The use as claimed in claim 9 wherein the antisense nucleic acid encodes one ore more ribozymes.

14. The use as claimed in many one of claims 1 to 3 wherein the nucleic acid encodes a therapeutic or prophylactic agent.

15. The use as claimed in claim 14 wherein the agent is an interferon-α subtype, eg. IFNα₈.

16. The use as claimed in claim 14 wherein the agent is an interleukin, eg. IL-12.

17. The use as claimed in any one of claims 4 to 16 wherein the nucleic acid includes one or more regulatory sequences capable of being activated in liver tumour cells but not in adult liver cells.

18. The use as claimed in claim 17 wherein the regulatory sequences include the promoter and enhancer elements from the alpha-foetoprotein gene.

19. The use as claimed in any one of claims 1 to 8 where the nucleic acid includes nucleic acid encoding Herpes simplex virus protein VP22.

20. The use as claimed in any one of claims 1 to 19 wherein the nucleic acid includes the coding sequence for creatinine kinase.

21. A baculovirus containing therapeutic and/or prophylactic nucleic acid.

22. A baculovirus as claimed in claim 21 modified by any one or more of the features of any one of claims 1 to 20.

23. The use of a baculovirus as defined in claim 21 or claim 22 in the manufacture of a medicament for the treatment or prophylaxis of an infection.

24. The use of a baculovirus as defined in claim 21 or claim 22 in the manufacture of a medicament for use in the prophylaxis or treatment of liver cancer.

25. A pharmaceutical formulation comprising a baculovirus as defined in claim 21 or claim 22 optionally together with one or more pharmaceutically acceptable carrier, excipients and/or diluents.

26. A method for the prophylaxis or treatment of an infection comprising administering to a subject an effective amount of a baculovirus as defined in claim 21 or claim 22.

27. A method for the propylaxis or treatment of liver cancer comprising administering to a subject an effective amount of a baculovirus as defined in claim 21 or claim 22.
